(19) United States
(12) Patent Application Publication
(10) Pub. No.: US 2013/0000019 A1
(43) Pub. Date: Jan. 3, 2013

(54) WORKING GLASSES

(75) Inventors: Wataru Okuma, Tokyo (JP); Kanako Ichikawa, Tokyo (JP)

(73) Assignee: MIDORI ANZEN CO., LTD., Tokyo (JP)

(21) Appl. No.: 13/583,800

(22) PCT Filed: Dec. 28, 2010
(86) PCT No.: PCT/JP2010/073788

§ 371 Date: Sep. 10, 2012

(30) Foreign Application Priority Data
Mar. 9, 2010 (JP) 2010-051704

(51) Int. Cl. A61F 9/02

(52) U.S. Cl. 2/443

(57) ABSTRACT

Grooves are formed in the inner surface of two rims, and a part of the inner side of each of the grooves is formed as a cutout section. Lenses are removed from the rims while avoiding the interference between a stopper member, which is pivotably provided to a bridge, and inner regions of the rims by pivoting downward the stopper member. Then, new lenses are inserted through the inner regions. After that, the stopper member is rotated upward and fixed to the bridge. As a result, the stopper member interferes with the inner regions of each of the rims to firmly fix the lenses within the rims.

### TECHNICAL FIELD

The present invention relates to working glasses in which two lenses attached to rims of a frame can be replaced by a simple method.

### BACKGROUND ART

Glasses for protecting eyes, which are used in a workplace, for example, have such problems that lenses may be scratched, soiled, or damaged during use, and if they are used in this state, it becomes hard to see through the glasses. Therefore, it is desirable to replace only the lenses by a simple method. As a conventional example of glasses that enables easy replacement of the lenses, glasses described in Japanese Patent Application Laid-Open No. 2002-214568 (Patent Literature 1) have been known.

FIG. 1 is an explanatory diagram showing the glasses disclosed in Patent Literature 1, in which thicknesses of an inner region and upper and lower regions of each rim 101 are large, and a groove 102 for fitting a lens 103 is formed in these portions. By inserting the lens 103 along the groove 102 from an outer region (denoted by reference sign A) of the rim 101, the lens can be attached into the rim 101. Further, fall of the lens 103 attached into the rim 101 is prevented by engaging an engagement protrusion 104 formed in the groove 102 with a notch 105 formed in the lens 103.

### PRIOR ART DOCUMENTS

### Patent Documents

Patent Document 1: Japanese Patent Application Laid-open No. 2002-214568

### DISCLOSURE OF INVENTION

### Technical Problem

However, the conventional example disclosed in Patent Literature 1 has such a configuration that the lens 103 attached into the groove 102 formed in each rim 101 is fixed only by engagement between the engagement protrusion 104 formed in the groove 102 and the notch 105 formed in the lens 103. Therefore, to firmly fix the lens 103 to the rim 101, a certain degree of friction force is required between the groove 102 and the lens 103.

Accordingly, when the lens 103 is attached into the groove, the lens 103 needs to be pressed with a rather strong force, thereby causing such a problem that the lens 103 may be scratched at the time of attachment or detachment of the lens 103, or the rim 101 may be deformed or damaged due to application of an excessive force to the rim 101.

The present invention has been achieved to solve these problems of the conventional technique, and an object of the present invention is to provide working glasses in which lenses can be easily attached or detached by a simple method and can be firmly fixed into rims.

### Technical Solution

To achieve the above object, a first aspect of the present invention is to provide working glasses comprising: a frame body including two rims and a bridge that connects the rims; and lenses to be fixed into the two rims, respectively, wherein a groove for fixing the lens to be attached to each of the rims is provided on an inner periphery of each rim; a cutout section is provided in a part on a central side of the frame body; and a stopper member that is provided to pivot around a direction connecting the rims and fixes the lenses by interference with the lenses attached to the grooves in the cutout section is provided to the bridge.

In a second aspect of the present invention depending on the first aspect, in the working glasses, the cutout section is formed by cutting the frame body on a front face side.

In a third aspect of the present invention depending on the first or second aspects, in the working glasses, a stepped portion is formed at an appropriate position of the groove formed in each rim; a notch is formed on a periphery of each lens in a portion that engages with the stepped portion; and after the lenses are attached to the grooves with a configuration mentioned above, the notches of the lenses are engaged with the stepped portions formed in the grooves, thereby fixing the lenses to the rims.

In a fourth aspect of the present invention depending on any of the first to third aspects, in the working glasses, the stopper member is pivotally attached to the bridge by a hinge member fixed to the bridge; and fixing and release of the lenses can be switched by pivoting the stopper member by the hinge member.

In a fifth aspect of the present invention depending on any of the first to fourth aspects, the working glasses further comprise a first protrusion formed in a lower region of one of the rims on a front face side thereof, a second protrusion formed in a lower region of the other of the rims on a front face side thereof, and a third protrusion formed on a front face of the stopper member; wherein when the frame body is placed on a plane with a front face side thereof facing downward with a configuration mentioned above, the first to third protrusions contact the plane to stabilize the working glasses on the plane.

### Advantageous Effects

According to the first aspect of the present invention described above, the lens is inserted into the rim from the cutout section formed on an inner side of each rim (on the central side of the frame body), and the lenses are fixed by the stopper member, thereby enabling to firmly fix the lenses attached into the rims. At this time, a large friction force is not generated between the grooves formed on the inner peripheries of the rims and the lenses. Therefore, a worker can attach or detach the lenses without requiring a strong force. Accordingly, occurrence of such a trouble that the lenses are scratched or the rims are deformed or damaged by applying an excessive force to the rims at the time of attachment of the lenses can be reduced. Further, because the lens can be inserted into the rim only by holding an end of the lens, a new one can be inserted without being soiled by hands (particularly, a central part of the lens). Furthermore, because the two lenses are fixed by one stopper member, the configuration can be simplified as compared to a case where the lenses are separately fixed.

According to the second aspect of the present invention described above, the cutout section is formed on the front face side of the frame body. Therefore, even if the lenses attached into the rims are detached due to a strong impact from outside, fall of the lenses on the side of the face of a wearer can be prevented.

According to the third aspect of the present invention described above, the notches formed on the lenses engage with the stepped portions formed in the grooves with the lenses being inserted into the rims. Therefore, fall of the lenses can be prevented before the lenses attached in the rims are fixed by the stopper member.

According to the fourth aspect of the present invention described above, the stopper member is pivotally supported on the bridge by using the hinge member. Therefore, separation of the stopper member from the frame body can be prevented, and occurrence of troubles such as loss of the stopper member can be avoided.

According to the fifth aspect of the present invention described above, the first and second protrusions are provided in the lower regions of the two rims, respectively, and the third protrusion is provided on the front face of the stopper member. Therefore, when the working glasses are placed on a plane, the three protrusions contact the plane and are stabilized. Consequently, the working glasses can be placed stably without scratching or soiling the lenses.

### BRIEF DESCRIPTION OF THE DRAWINGS

FIG. 1 is an explanatory diagram showing a configuration of conventional glasses with interchangeable lenses.

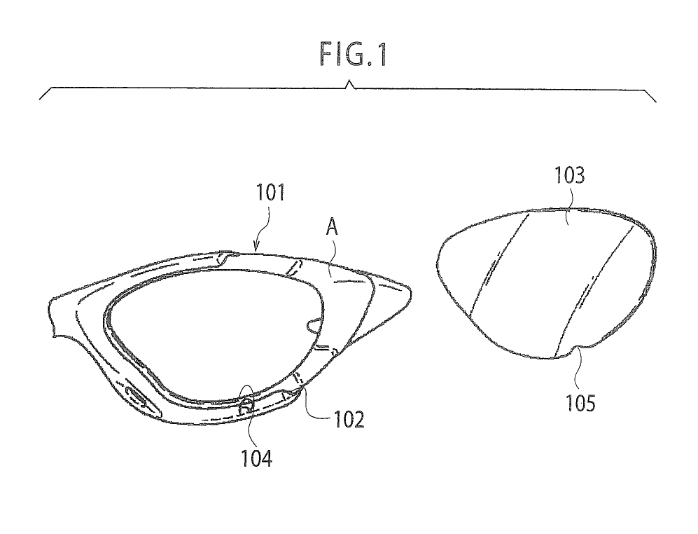

FIG. 2 is a perspective view showing an overall configuration of working glasses according to a first embodiment of the present invention, and shows a state where lenses are fixed by a stopper member.

FIG. 3 is a perspective view showing an overall configuration of the working glasses according to the first embodiment, and shows a state where the stopper member is released.

FIG. 4 is a perspective view showing an overall configuration of the working glasses according to the first embodiment, and shows a state where the lenses are detached.

FIG. 5 is an explanatory diagram of the working glasses according to the first embodiment, and is a side view of the working glasses when a frame body is placed on a plane with a front face thereof facing downward.

FIG. 6 is an explanatory diagram of the working glasses according to the first embodiment, and is a top view of the working glasses when the frame body is placed on a plane with the front face facing downward.

FIG. 7 is a perspective view showing an overall configuration of working glasses according to a second embodiment of the present invention, and shows a state where lenses are fixed by a stopper member.

FIG. 8 is a perspective view showing an overall configuration of the working glasses according to the second embodiment, and shows a state where the stopper member is released.

FIG. 9 is a perspective view showing an overall configuration of the working glasses according to the second embodiment, and shows a state where the lenses are detached.

FIG. 10 is an explanatory diagram showing a state where an oil drop adhered to a lens surface of the working glasses according to the first embodiment is falling downward.

FIG. 11 is an explanatory diagram of the working glasses according to the first embodiment, and is an explanatory diagram when each rim is vertically divided into two.

### BEST MODES FOR CARRYING OUT THE INVENTION

Embodiments of the present invention are explained below with reference to the drawings.

### First Embodiment

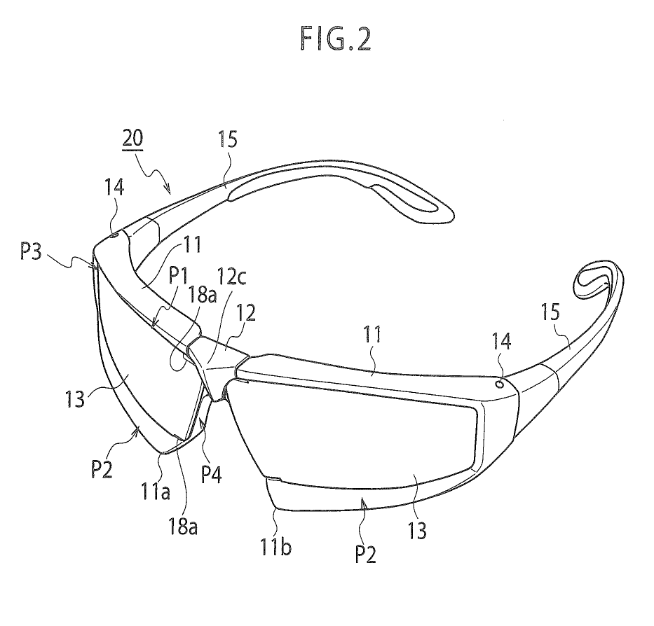

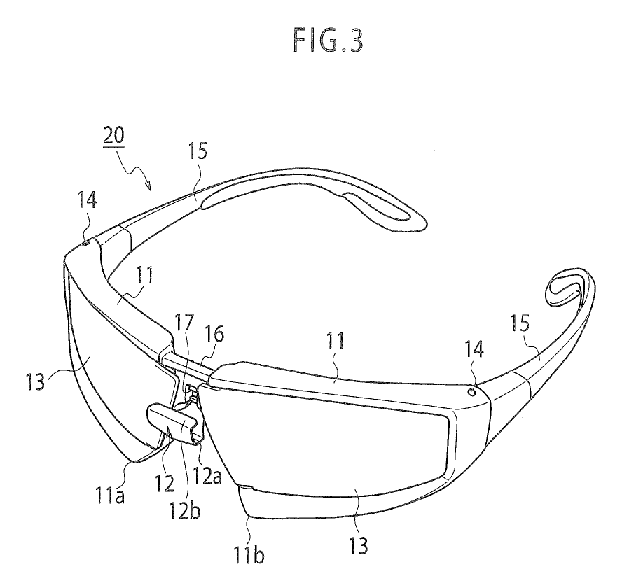

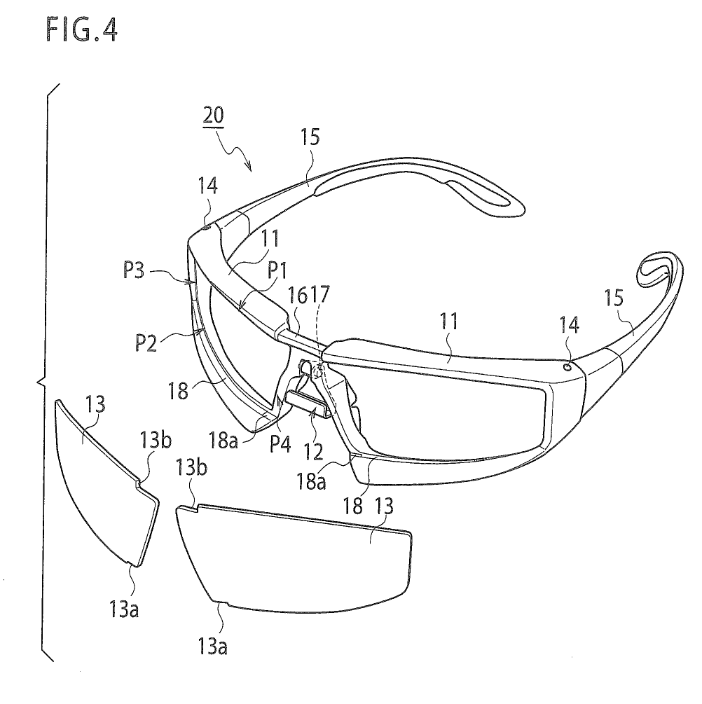

FIGS. 2 to 4 are perspective views showing a configuration of working glasses 20 according to a first embodiment of the present invention. FIG. 2 shows a state where lenses 13 are attached into rims 11, FIG. 3 shows a state where a stopper member 12 is released, and FIG. 4 shows a state where the lenses 13 are detached from the rims 11.

As shown in FIGS. 2 to 4, the working glasses 20 include two rims 11, and the rims 11 are connected by a bridge 16 (see FIGS. 3 and 4). The two rims 11 and the bridge 16 constitute a frame body of the working glasses according to the present invention. A temple 15 (also referred to as “bow”) is attached to an outer end of each rim 11 by a hinge 14.

The temples 15 are placed over the ears when a worker wears the working glasses 20, and can be folded by turning the temples 15 with respect to the rims 11 on the hinges 14.

As shown in FIG. 4, a groove 18 for attaching the lens 13 is formed in an upper region P1, a lower region P2, and an outer region P3 of an inner periphery of each of the rims 11. An inner region P4 of the inner periphery of the rim 11 is open on a front face side thereof (an opposite side to the worker's face). That is, the inner region P4 of the rim 11 is cut out on the front face side, and the groove 18 is not formed in this region (a cutout section). The lens is attached and detached from the open region.

Furthermore, stepped portions 18a (see FIGS. 2 and 4) are formed at ends on the central side of the lower region P2 and the upper region P1 of the groove 18 formed in each rim 11. Notches 13a and 13b are respectively formed in lower and upper sides of the lens 13 made of plastic or glass to be attached into each rim 11 (see FIG. 4). When the lens 13 is attached into the rim 11, the notches 13a and 13b engage with the stepped portions 18a formed in the groove 18 to prevent fall of the lens 13 from the rim 11. A configuration where the notches 13a and 13b and the stepped portions 18a are not provided can be also used.

Meanwhile, as shown in FIGS. 3 and 4, the stopper member 12 with a hook-shaped cross section is provided to the bridge 16 connecting the two rims 11, and the stopper member 12 is pivotally supported on a lower side of the bridge 16 by a hinge 17 (hinge member). Accordingly, the stopper member 12 can pivot upward and downward around the hinge 17 as a rotation shaft. As shown in FIGS. 3 and 4, when the stopper member 12 is pivoted downward, the stopper member 12 does not interfere with the inner regions P4 of the rims 11, thereby enabling to attach or detach the lenses 13. When the stopper member 12 is pivoted upward, a concave portion 12a (see FIG. 3) of the stopper member 12 fits to the bridge 16 to fix the stopper member 12 to the bridge 16. At this time, the stopper member 12 interferes with the inner regions P4 of the rims 11, and inner sides of the lenses 13 contact the stopper member 12, thereby fixing the lenses 13.

Furthermore, as shown in FIG. 2, the lower regions P2 of the rims 11 protrude forward from outer sides to inner sides thereof, and inner ends thereof become a protrusion 11a (first protrusion) and a protrusion 11b (second protrusion), respectively. A protrusion 12c (third protrusion) is formed on the front face side of the stopper member 12 (an opposite side to the worker's face). The working glasses 20 can be stably placed on a work table or the like because of the protrusions 11a, 11b, and 12c as described later.

A procedure of attaching and detaching the lenses 13 in the working glasses 20 according to the first embodiment constituted as described above is explained next.

As shown in FIG. 4, the lenses 13 are attached to the rims 11, to which the lenses 13 are not attached yet. In this operation, the lenses 13 are attached by gradually sliding the lenses from the side of the inner regions P4 of the rims 11 to cause the upper sides and the lower sides of the lenses 13 to follow the grooves 18 formed in the rims 11. At this time, sliding friction between the lenses 13 and the grooves 18 is small, and the worker can easily attach the lenses 13 into the rims 11 without applying a strong force.

When the lenses 13 are attached into the rims 11, the stepped portions 18a of the grooves 18 properly engage with the notches 13a and 13b formed in the lenses 13, thereby fixing the lenses 13 into the rims 11. In this state, the lenses 13 have slight backlashes with respect to the rims 11. However, fall of the lenses 13 from the rims 11 is prevented by engagement between the notches 13a and 13b and the stepped portions 18a. That is, even if the working glasses 20 are tilted upward or downward, or rightward or leftward in this state, the lenses 13 do not fall out of the rims 11.

The stopper member 12 is then rotated on the hinge 17 as an axis, and when an end portion 12b as shown in FIG. 3 is elastically deformed slightly to pass over the bridge 16, the concave portion 12a of the stopper member 12 fits to the bridge 16, thereby fixing the stopper member 12 to the bridge 16 (see FIG. 2). In this state, the stopper member 12 interferes with the inner regions P4 of the rims 11 to firmly fix the lenses 13 attached into the rims 11, thereby enabling to prevent fall of the lenses 13.

When the lenses 13 are to be detached, it suffices to perform a reverse operation to that mentioned above. That is, when the stopper member 12 is rotated forward from the state where the stopper member 12 engages with the bridge 16 to fix the lenses 13 (the state shown in FIG. 2), interference of the stopper member 12 with the inner regions P4 of the rims 11 is released (the state shown in FIG. 3).

When the stopper member 12 is further rotated to a position shown in FIG. 4, the lenses 13 can be easily detached from the rims 11 by sliding the lenses 13 inward of the frame body, and the lenses can be replaced by new ones.

In this way, the working glasses 20 according to the first embodiment has such a configuration that the inner region P4 of each rim 11 is formed as the cutout section and the lens 13 is inserted into the rim 11 from the inner region P4, and the stopper member 12 is fitted to the bridge 16, thereby enabling to firmly fix the lenses 13 attached into the rims 11.

Further, because the lenses 13 are not fixed to the rims 11 by the friction force between the grooves 18 and the lenses 13 as in the conventional example, any strong force is not required at the time of sliding the lenses 13 along the grooves 18, thereby enabling to reduce occurrence of such a trouble that the lenses 13 are scratched or the rims 11 are deformed or damaged by applying an excessive force to the rims 11. Because the lens 13 can be inserted into the rim 11 only by holding an end of the lens 13, a new lens can be inserted without soiling the new lens (particularly, the central part of the lens) by hands.

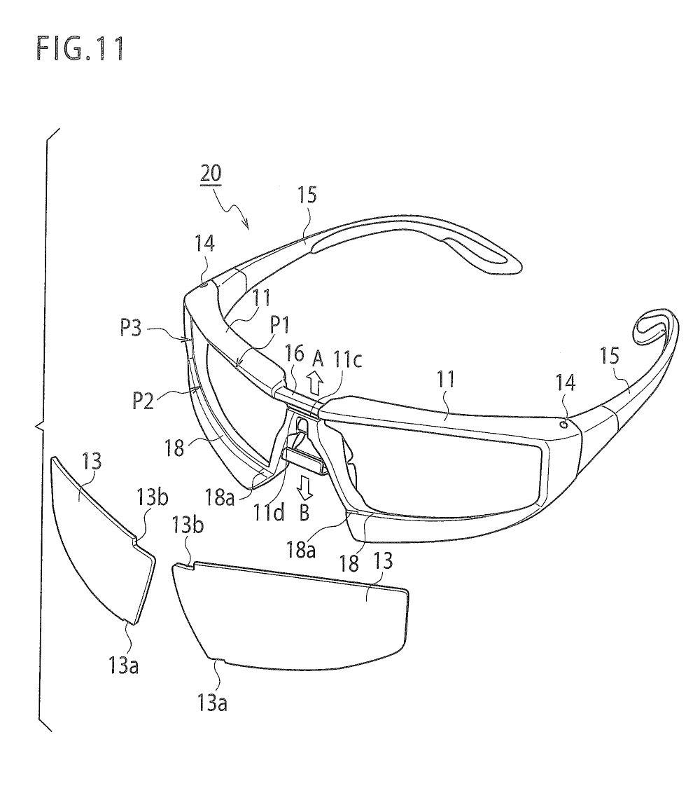

When the central part of the rim 11 (for example, a part under the bridge 16) is vertically divided into two as shown in FIG. 11, the lens 13 can be replaced more easily. In FIG. 11, when the rim 11 is elastically deformed to separate an upper rim 11c and a lower rim 11d away from each other by pulling the upper rim 11c upward (in a direction shown by an arrow A) and the lower rim 11d downward (in a direction shown by an arrow B), the rim 11 is opened vertically, and insertion or pullout of the lens 13 into or from the rim 11 becomes more smooth.

The notches 13a and 13b formed in the lens 13 engage with the stepped portions 18a formed in the grooves 18 with the lenses 13 being inserted, thereby enabling to prevent fall of the lenses 13 before the lenses 13 are fixed by the stopper member 12.

Furthermore, the two lenses 13 are fixed by one stopper member 12, and thus the configuration can be simplified as compared to a case where the lenses 13 are fixed separately. Because the stopper member 12 is pivotally supported on the bridge 16 by the hinge 17, the stopper member 12 is not detached from the frame body, and occurrence of troubles such as loss of the stopper member 12 can be avoided. The stopper member 12 needs not have the configuration of being pivotally supported on the bridge 16 by the hinge 17, and can have any configuration so long as the stopper member 12 is pivotally supported.

The inner regions P4 of the inner peripheries of the rims 11 on the front face side thereof are open to insert the lenses 13. Accordingly, even if the lenses 13 attached into the rims 11 are detached due to a strong impact from outside, fall of the lenses on the side of the wearer's face or worker's discomfort can be prevented.

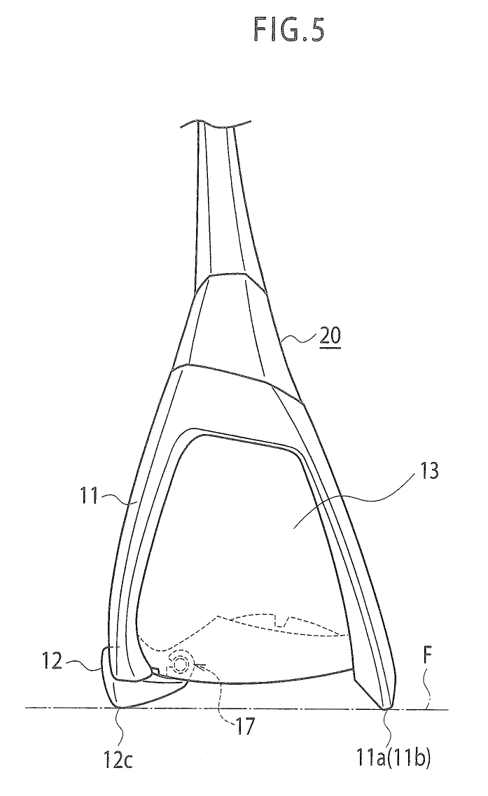

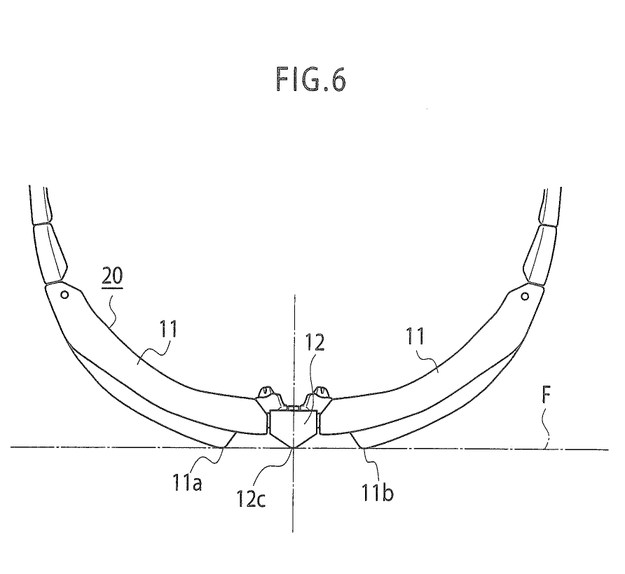

As shown in FIG. 2, the protrusions 11a and 11b are formed in the lower regions P2 of the rims 11 and the protrusion 12c is formed on the stopper member 12. Therefore, when the glasses 20 are placed on a plane such as a work table with the front face of the working glasses 20 being downward, three points of the protrusions 11a, 11b, and 11c contact the plane to stabilize the glasses, and thus the working glasses 20 can be placed on the work table or the like in a stable state without scratching or soiling the lenses 13. That is, FIG. 5 is a side view when the working glasses 20 according to the first embodiment is placed on a plane F with the front face facing downward, and FIG. 6 is a top view thereof. As shown in FIGS. 5 and 6, because only the three points of the protrusions 11a, 11b, and 11c contact the plane F, the lenses 13 do not contact the plane F, and the working glasses 20 can be stably placed on the plane F.

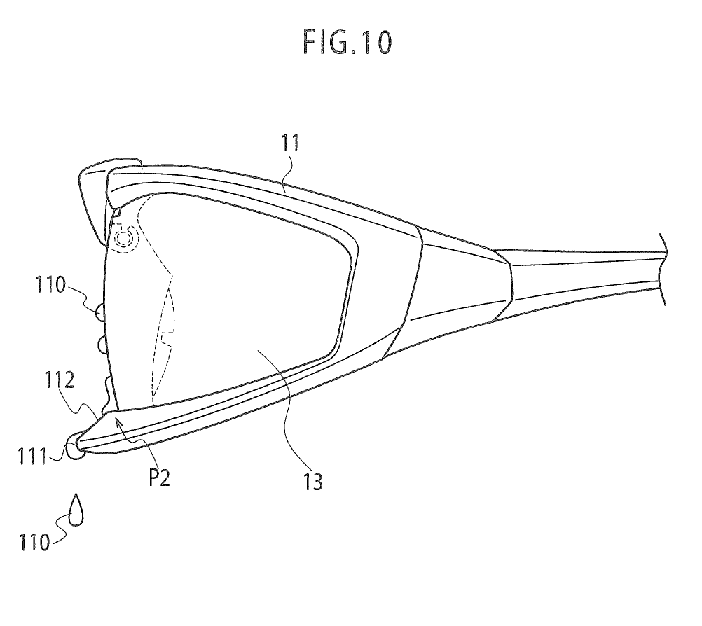

Furthermore, as shown in FIG. 10, the lower regions P2 of the rims 11 protrude along the front face side and a front face 112 thereof tilts downward and forward (in a direction away from the face). Accordingly, for example, when the worker is wearing the working glasses 20 and an oil drop (or dust) 110 adheres to the surface of the lens 13 and drips downward, the oil drop 110 falls onto a protruded part 111 and flows down forward, and thus direct dripping of the oil drop onto the worker's face can be prevented, thereby improving the workability.

### Second Embodiment

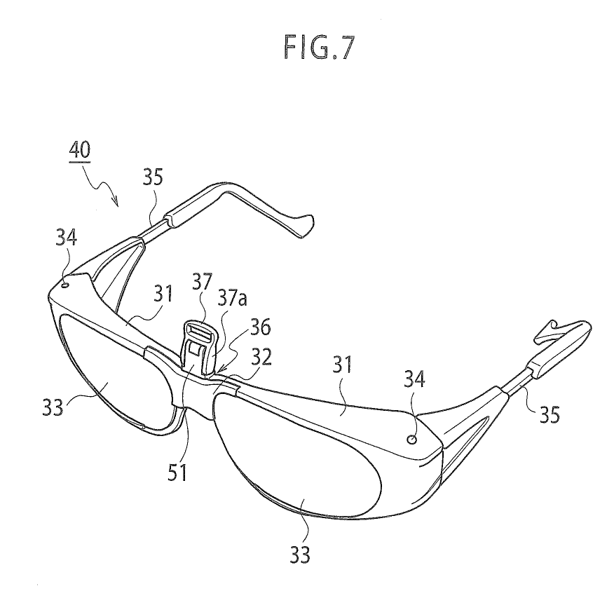

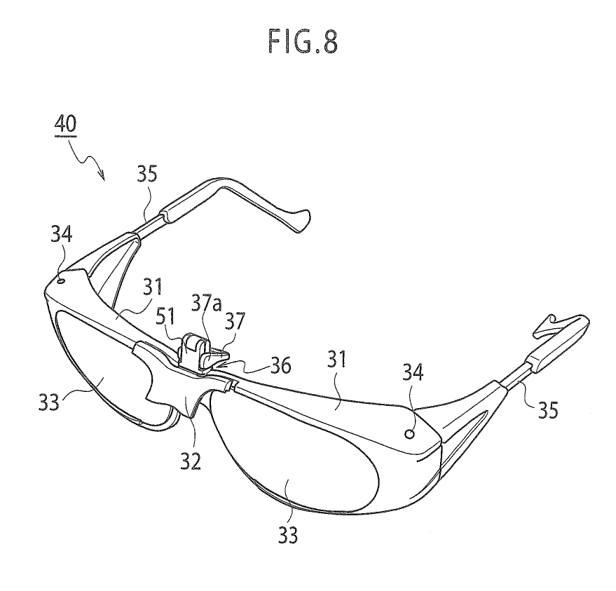

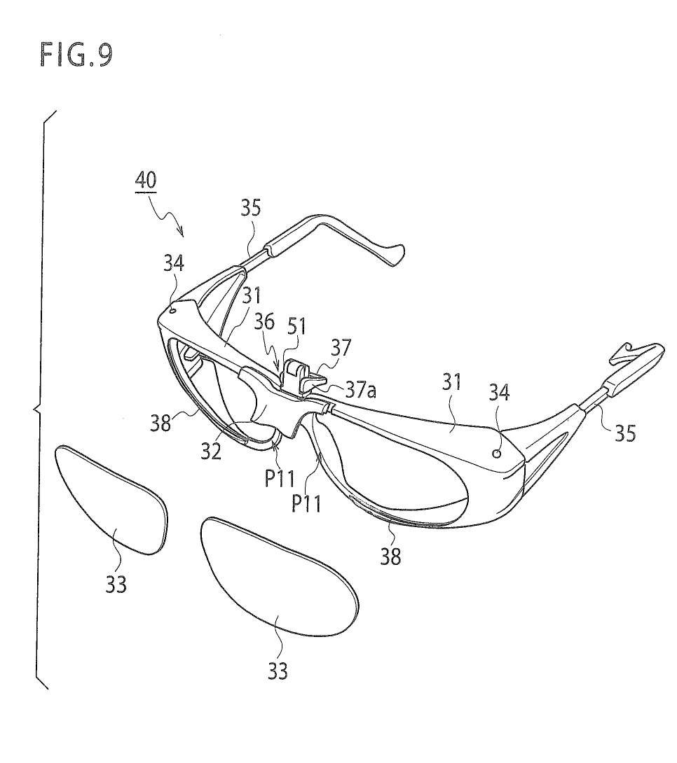

Working glasses according to a second embodiment of the present invention are explained next. FIGS. 7 to 9 are perspective views showing a configuration of working glasses 40 according to the second embodiment. FIG. 7 shows a state where lenses 33 are attached into rims 31. FIG. 8 shows a state where a stopper member 32 is released, and FIG. 9 shows a state where the lenses 33 are detached from the rims 31.

As shown in FIGS. 7 to 9, the working glasses 40 include two rims 31, and the rims 31 are connected by a bridge 36. The two rims 31 and the bridge 36 constitute a frame body of the working glasses according to the present invention. Furthermore, a temple 35 is attached to an outer end of each rim 31 by a hinge 34. The temples 35 are placed over the ears when a worker wears the working glasses 40, and can be folded by turning the temples 35 with respect to the rim 31 on the hinge 34.

As shown in FIG. 9, a groove 38 for attaching the lens 33 is formed in a region of an inner periphery of each rim 31 except for an inner region P11. The inner region P11 of the inner periphery of the rim 31 is open on a front face side thereof (an opposite side to the worker's face). That is, the inner region P11 of the rim 31 is cut out on the front face side of the rim 31, and the groove 38 is not formed in this region (a cutout section). The lens 33 is attached and detached from the open inner region P11.

Meanwhile, a support member 51 for supporting swing of the stopper member 32 is provided on the bridge 36, and an operating member 37 is pivotally supported by the support member 51. That is, a shaft 37a of the operating member 37 is pivotally supported by the support member 51 and thus the operating member 37 can be pivoted vertically.

The operating member 37 is connected to the stopper member 32 by a link mechanism (detailed explanations thereof will be omitted) and the stopper member 32 pivots together with vertical movement of the operating member 37. That is, when the operating member 37 is pivoted downward, the stopper member 32 pivots upward in association therewith (the state shown in FIG. 8), and when the worker turns the operating member 37 upward to pass a predetermined dead point, the stopper member 32 pivots downward (the state shown in FIG. 7).

An attachment and detachment procedure of the lenses 33 in the working glasses 40 according to the second embodiment configured as described above will be explained next.

As shown in FIG. 9, when the lenses 33 are not attached yet, the operating member 37 is laid down, and the stopper member 32 that operates together with the operating member 37 is raised upward. Accordingly, the stopper member 32 does not interfere with the inner regions P11 of the rims 31.

In this state, as shown in FIG. 9, the worker attaches the lenses 33 to the rims 31 to which the lenses 33 are not attached. In this operation, the lenses 33 are attached by gradually sliding the lenses from the side of the inner regions P11 of the rims 31 to cause the upper sides and the lower sides of the lenses 33 to follow the grooves 38 formed in the rims 31. At this time, sliding friction between the lenses 33 and the grooves 38 is small, and the worker can easily attach the lenses 33 into the rims 31 without applying a strong force.

When the operating member 37 is vertically pivoted to pass the predetermined dead point after attachment of the lenses 33, the operating member 37 stands upright, and the stopper member 32 pivots downward together with the operating member 37 to interfere with the inner regions P11 of the rims 31 as shown in FIG. 7. Therefore, the lenses 33 attached into the rims 31 are firmly fixed by the stopper member 32. Accordingly, fall of the lenses 33 from the rims 31 can be prevented.

Meanwhile, when the lenses 33 are to be detached from the rims 31, it suffices to perform a reverse operation to that mentioned above. That is, when the operating member 37 is pivoted downward to pass the dead point, the stopper member 32 pivots upward in association therewith to open the inner regions P11 of the rims 31. The lenses 33 can be easily detached from the rims 31 by sliding the lenses 33 inward of the frame body in this state, and the lenses 33 can be replaced by new ones.

Also in the working glasses 40 according to the second embodiment as in the working glasses 20 according to the first embodiment, the inner regions P11 of the rims 11 are opened to insert the lenses 33 into the rims 11 from the inner regions P11. The stopper member 32 is then pivoted downward to cause the stopper member 32 to interfere with the inner regions P11 and accordingly the lenses 33 attached into the rims 31 can be firmly fixed.

Because any strong force is not required when the lenses 33 are slid along the grooves 38 formed on the inner peripheries of the rims 31, occurrence of such a trouble that the lenses 33 are scratched or the rims 31 are deformed or damaged by applying an excessive force to the rims 31 can be reduced at the time of attachment and detachment of the lenses 33.

Furthermore, because the two lenses 33 are fixed by one stopper member 32, the configuration can be simplified as compared to a case where the lenses 33 are fixed separately. Further, because the stopper member 32 is pivotally supported by the support member 51 and swings vertically, the stopper member 32 is not detached from the frame body, and occurrence of troubles such as loss of the stopper member 32 can be avoided.

While the working glasses of the present invention have been explained based on the embodiments shown in the drawings, the present invention is not limited thereto and configurations of respective elements can be replaced by arbitrary ones having similar functions.

The entire contents of Japanese Patent Application No. 2010-051704 (filed on Mar. 9, 2010) are incorporated by reference in the specification of the present application.

### INDUSTRIAL APPLICABILITY

The present invention can be applied to working glasses for protecting workers' eyes during works performed in a manufacturing factory, a construction site and the like, and the working glasses are highly useful when lenses attached to a frame body are replaced in a simple operation.

1. Working glasses comprising:
a frame body including two rims and a bridge that connects the rims; and
lenses to be fixed into the two rims, respectively, wherein
a groove for fixing the lens to be attached to each of the rims is provided on an inner periphery of each rim;
a cutout section is provided in a part on a central side of the frame body; and
a stopper member that is provided to pivot around a direction connecting the rims and fixes the lenses by interference with the lenses attached to the grooves in the cutout section is provided to the bridge.

2. The working glasses according to claim 1, wherein the cutout section is formed by cutting the frame body on a front face side.

3. The working glasses according to claim 2, wherein
a stepped portion is formed at an appropriate position of the groove formed in each rim;
a notch is formed on a periphery of each lens in a portion that engages with the stepped portion; and
after the lenses are attached to the grooves with a configuration mentioned above, the notches of the lenses are engaged with the stepped portions formed in the grooves, thereby fixing the lenses to the rims.

4. The working glasses according to claim 3, wherein
the stopper member is pivotally attached to the bridge by a hinge member fixed to the bridge; and
fixing and release of the lenses can be switched by pivoting the stopper member by the hinge member.

5. The working glasses according to claim 4, further comprising:
a first protrusion formed in a lower region of one of the rims on a front face side thereof, a second protrusion formed in a lower region of the other of the rims on a front face side thereof, and a third protrusion formed on a front face of the stopper member; wherein
when the frame body is placed on a plane with a front face side thereof facing downward with a configuration mentioned above, the first to third protrusions contact the plane to stabilize the working glasses on the plane.
